(19) United States
(12) Patent Application Publication
(10) Pub. No.: US 2025/0268672 A1
(43) Pub. Date: Aug. 28, 2025

(54) HANDLING DEVICE FOR HANDLING MEDICAL AND/OR SURGICAL INSTRUMENTS, AND MEDICAL SYSTEM HAVING A HANDLING DEVICE

(71) Applicant: KARL STORZ SE & Co. KG, Tuttlingen (DE)

(72) Inventors: Martin HOFFMANN, Tuttlingen (DE); Pascal SCHULZKI, Tuttlingen (DE); Ulrich GRUHLER, Tuttlingen (DE)

(73) Assignee: KARL STORZ SE & Co. KG, Tuttlingen (DE)

(21) Appl. No.: 18/704,888

(22) PCT Filed: Oct. 13, 2022
(86) PCT No.: PCT/EP2022/078586

§ 371 Date: Apr. 25, 2024

(30) Foreign Application Priority Data
Oct. 26, 2021 (DE) 10 2021 127 877.5

(51) Int. Cl. A61B 34/37; A61B 17/00; A61B 90/00; A61B 90/90; G10L 17/22; H04R 1/02; H04R 1/08

(52) U.S. Cl. CPC A61B 34/37; A61B 17/00; A61B 90/361; A61B 90/90; G10L 17/22; A61B 2017/00203; H04R 1/028; H04R 1/086

(57) ABSTRACT

A handling device for handling a medical and/or surgical instrument, includes a multi-joint robot arm, which at its distal end carries an instrument holder designed for holding a medical and/or surgical instrument, and a control device, which is designed at least to control the multi-joint robot arm, wherein the handling device includes a speech detection device with at least one microphone, which is arranged in a region of the distal end of the multi-joint robot arm or integrated therein and is designed to detect at least a voice of a user of the handling device.

### CROSS-REFERENCE TO RELATED APPLICATIONS

This application is the U.S. national stage of PCT/EP2022/078586 filed on Oct. 13, 2022, which claims priority of German Patent Application No. 10 2021 127 877.5 filed on Oct. 26, 2021, the contents of which are incorporated herein.

### TECHNICAL FIELD

The present disclosure relates to a handling device for handling medical and/or surgical instruments, according to the preamble of claim 1. The present disclosure further relates to a medical system comprising such a handling device and a medical and/or surgical instrument.

### BACKGROUND

Handling devices of the type in question, for handling medical and/or surgical instruments, are generally known from the prior art and are already extensively used in the performance of operations.

During an operation, it is also desirable, for some applications, to have a means of sound or speech detection, for example in order to detect the voice of the operating surgeon and/or of members of the operating team. This may be necessary, for example, if the surgeon wishes to contact one or more colleagues by telephone during the operation, for example in order to consult with them regarding the operation. Similarly, sound capture of this kind may be advantageous for conducting training events in which the surgeon concerned explains his or her procedure during the operation. In addition, sound capture of this kind during the operation may be advantageous for voice control of medical and/or surgical instruments or the like, since such voice control does not require manual intervention of a person, and therefore the surgeon concerned can fully concentrate on the manual performance of the operation.

A problem with known sound capture or speech detection is that, in a surgical environment, there are usually a great many acoustic interference sources within the operating room, which makes speech detection difficult. Such sources of acoustic interference are, for example, instruments for recording vital parameters of the patient, operating instruments and/or ventilators.

To avoid the influence of these sources of acoustic interference on speech detection, it is known from the prior art to place at least one recording microphone in proximity to the surgeon or the voice source. However, care must be taken to ensure that this placement does not negatively affect the ergonomics of the surgeon or impact his or her working environment. For example, it is known for the surgeon concerned or for a group of surgeons each to wear a headset, a clip-on microphone and/or a headphone with an integrated microphone. Such placement of the microphone is advantageous for voice capture, since the microphone is placed in direct proximity to the voice source. However, microphones arranged in this way can cause ergonomic disturbance, which can be perceived as a nuisance by the surgeon, especially during protracted operations. Moreover, the fitting of portable microphones of this kind entails additional work in ensuring the sterility of the operating field and that of the surgeon. A particular challenge lies in sterilization of the used microphone itself, since the latter has to be technically designed and suitable for the sterilization (possibly by UV light or similar).

Such a medical system is known, for example, from U.S. Pat. No. 7,395,249 B1. The system is used in a sterile operating room where a surgeon performs a surgical procedure on a patient. The patient lies on an operating table to which a multi-joint robot arm is attached that is able to move a surgical instrument relative to the table and to the patient. The surgical instrument is, for example, an endoscope, which is inserted for example into the abdomen of the patient. The individual joints of the multi-joint robot arm can be controlled by respective drives, thus ensuring a multi-dimensional mobility of the robot arm relative to the patient and/or to the surgeon. The system has a microphone connected to a computer and worn as a headset by the surgeon. By placing the microphone in direct proximity to the surgeon, the amount of background noise can be decreased and the likelihood of an accidental or incorrect input command can be reduced.

Furthermore, a system for monitoring a medical procedure performed in a clinical environment is known from U.S. Pat. No. 10,537,291 B2. Such a system comprises an audio recorder which is configured to generate a data signal representative of verbal communication by the surgeon concerned. Furthermore, the known system can comprise a sound recorder with at least one microphone, which can be worn, for example, as a headset by the surgeon.

Alternatively, it is known in the prior art to arrange one or more microphones in devices in the periphery of the surgeon within the operating room and, if necessary, to filter out noise interference through acoustic filters. Such microphones can be provided, for example, in what are called device traffic lights or in device trolleys, which however do not follow a (head) movement of the operating surgeon during the operation. This type of arrangement offers good ergonomics for the surgeon, since he or she does not have to wear any additional equipment. However, during the operation, the distance between the microphone and the voice source may change continuously, which can lead to a loss of recording quality, so that the latter is suboptimal and/or susceptible to interference depending on the distance. Furthermore, the fixed microphones are often arranged outside the sterile region in the operating room in order to avoid the problems of sterilization, but the resulting additional distance leads to further challenges as regards the quality of the speech detection.

The problems surrounding the sterilization of portable microphones have already been highlighted, for example in U.S. Pat. No. 10,617,400 B2, and a corresponding solution proposed. The solution is to provide a large number of microphones in an operating room in order to eliminate the need for portable headsets. The noises detected by the microphones are separated from one another by a sound source separation unit and are assigned to a respective sound source on the basis of position information regarding the respective position of the microphones in the operating room. As a result of the sound separation, separate sound information is obtained for each sound source. It is also necessary to remove interference from the detected noises by means of acoustic filtering, as a result of which the system appears technically complex.

Furthermore, WO 2020/040795 A1 discloses a surgical instrument, for example an endoscope, which comprises several microphones attached to an instrument housing. The surgical instrument can form a sterile barrier (for example in the manner of a drape). This barrier can be arranged facing an environment within the operating room in such a way that the surgeon can speak into the microphone at the operating table. WO 2020/040795 A1 has also highlighted the problem that the use of headsets can interfere with the surgical workflow in the operating room, and that microphones placed at different locations in the operating room, e.g. hanging from the ceiling, may not reach as far as the region between the operating table and the surgeon, such that the speech detection is not sufficiently guaranteed. The solution proposed in WO 2020/040795 A1 is a surgical robot system having a communication device. A microphone is integrated directly in the instrument, which can be arranged on the robot arm. The instrument can be an endoscope, a sterile barrier, a cystoscope, a laparoscope, an arthroscope or a sterile adapter, for example.

Although the solution approach known from WO 2020/040795 A1 contributes in principle to remedying the abovementioned problems as regards the placement of microphones for speech detection, this solution approach also does not appear sufficient. For example, the problem with arranging microphones in surgical instruments is that these instruments need to be thoroughly sterilized after each operation. The arrangement of the microphones on the tools means that they too must be sterilized, as a result of which the microphones have to be particularly robust. In addition, constant sterilization has a negative impact on the useful life of the microphones. These instruments are also replaceable components that may need to be exchanged during an operation. Therefore, in order to ensure comprehensive speech detection, it appears necessary to provide the microphones on all of the required instruments. This increases the costs of producing the instruments concerned.

It is therefore an object of the present disclosure to overcome the aforementioned disadvantages of the prior art. In particular, an object is to improve the sterilizability of a microphone in the operating environment and and to improve the quality of speech detection or sound capture compared to the prior art.

Moreover, the object is to provide a medical system having at least one handling device according to the disclosure and a medical and/or surgical instrument.

### SUMMARY

The abovementioned object is achieved by a handling device having the features of claim 1. Moreover, the object is achieved by a medical system according to the disclosure.

Advantageous developments of the disclosure are set forth in the dependent claims. The scope of the disclosure covers all combinations of at least two of the features disclosed in the description, the claims and/or the figures.

In the context of the present disclosure, a handling device is provided for handling at least one medical and/or surgical instrument. The handling device according to the disclosure comprises a multi-joint robot arm which at its distal end carries an instrument holder. The instrument holder is designed to hold or carry a medical and/or surgical instrument. Furthermore, the handling device comprises a control device which is designed at least to control the multi-joint robot arm. The handling device according to the disclosure is characterized in that it comprises a speech detection device with at least one microphone, which is arranged in a region of the distal end of the multi-joint robot arm and is designed to detect at least a voice of a user of the handling device. Alternatively or in addition, the at least one microphone can also be integrated in a region of the distal end of the multi-joint robot arm.

Compared to the prior art, the handling device according to the disclosure offers the advantage that at least the at least one microphone of the speech detection device, by virtue of its arrangement at the distal end of the multi-joint robot arm, is always located near the head of the user or of the surgeon. If the user speaks during the operation, his or her mouth is also in direct proximity to the at least one microphone. This allows the user's voice to be detected as a sound source by the microphone in close proximity to the mouth, thereby minimizing the influence of acoustic noise interference from the user's periphery (e.g. caused by further devices located in the operating room). Thus, it is no longer absolutely necessary for the signals detected by the microphone to be filtered by means of acoustic filters in order to free the signals from noise interference, since the captured voice of the user, on account of his or her (permanent) proximity to the at least one microphone, is in any case detectable almost without any interference.

A further advantage of the handling device according to the disclosure is that the at least one microphone is arranged and/or integrated directly in the region of the distal end of the multi-joint robot arm. Compared in particular to the arrangement of a microphone known from WO 2020/040795 A1 on a medical and/or surgical instrument, this arrangement according to the disclosure has the advantage that, independently of the instrument held on the handling device, a microphone is at all times provided in the distal end region of the multi-joint robot arm. Thus, in order to provide speech detection before, during and after an operation, it is no longer necessary to equip all the instruments concerned with a microphone. Rather, the at least one microphone is arranged on the end of the handling device or of the multi-joint robot arm.

By virtue of this inventive arrangement of the at least one microphone, a further advantage is that of simplifying a medically required sterilization of the operating environment and in particular of the instruments used. Following their use, the handling device and the microphone can thus be sterilized separately from the instrument used. The instruments used, on the other hand, can be removed from the handling device and cleaned or sterilized separately. In particular, compared to WO 2020/040795 A1, the resulting advantage is that the instruments can be sterilized independently of any necessary sterilization of the at least one microphone, which is arranged on the multi-joint robot arm.

The handling device according to the disclosure is thus provided, in its intended use, for holding or carrying a medical and/or surgical instrument which is used, for example, during an operation. A handling device of the type in question for holding and/or handling medical and/or surgical instruments is disclosed, for example, in EP 3 753 519 A1 from the applicant, the technical teaching of which document is included in its entirety in the present application.

Handling devices according to the disclosure, and generic handling devices too, provide for example a motorized, controllable holding arm (multi-joint robot arm), which is used in particular in open and/or microsurgical procedures on the human or animal body, for example in the fields of neurosurgery and spinal surgery, and in ear nose and throat surgery. Such holding arms ensure an ergonomic and comfortable posture of the operating surgeon by virtue of the flexible positioning of the holding arm. It is possible, for example, to predefine a plurality of approach positions of the holding arm in order to retrieve these positions during the operation. In addition, such holding arms offer the possibility of combining the microscopy and endoscopy applications that are often required during surgery. This is known, for example, from the field of endoscope-assisted microsurgery (EAM).

It will be appreciated that the handling device according to the disclosure can comprise more than one microphone (at least one microphone). The microphones can in principle be arranged and/or integrated at different locations in the region of the distal end of the multi-joint robot arm.

The control device is designed at least to control the multi-joint robot arm, but it can also be designed to control at least one medical and/or surgical instrument. In principle, the control device for this purpose can have different control units or a common control unit, which can be integrated in the handling device or arranged separately from the latter.

The instrument holder preferably forms an end effector of the multi-joint robot arm, to which the at least one instrument can preferably be fastened reversibly in a releasable or detachable manner. The instrument holder preferably forms a kind of connection platform, on which a wide variety of medical and/or surgical instruments can be held.

The expression “to detect at least a voice of a user of the handling device” is understood here as meaning that the speech detection device is designed to detect the voice (i.e. speech and/or other sounds, i.e. acoustic signals) of at least one user, usually a surgeon. The speech detection device can comprise further components; the at least one microphone can in principle be arranged and/or integrated on the multi-joint robot arm separately from the rest of the speech detection device. However, the speech detection device can also be arranged and/or integrated in its entirety on the multi-joint robot arm. The speech detection device can further comprise an (internet) telephone unit, which is designed to transmit the detected speech as a long-distance call. The speech detection device can further comprise an acoustic output unit, for example a loudspeaker, which in principle can be arranged separately from the at least one microphone. The speech detection device can further comprise an acoustic recording and storage unit, which is designed to record and store the captured acoustic signals in a reproducible form.

The expression “in the region of the distal end of the multi-joint robot arm” is understood here as meaning that the at least one microphone is preferably arranged on the multi-joint robot arm, in particular at the free (freely movable) end thereof when considered along a kinematic path of the multi-joint robot arm.

The expression “integrated therein” is understood here as meaning that the at least one microphone can be introduced, for example, into a section of a joint arm of the multi-joint robot arm or into a joint itself, or can be arranged below an outer surface of the multi-joint robot arm.

In a preferred embodiment, the handling device comprises a platform from which the multi-joint robot arm extends. The control device is furthermore designed to move at least the distal end of the multi-joint robot arm relative to the platform (preferably in several degrees of freedom). For example, the platform can be provided in the form of a base or pedestal. Similarly the platform can be provided in the manner of a (preferably mobile) carriage (for example, as the support side thereof facing away from the floor). Such a carriage can be placed, for example, near an operating table and can be fixed in position (e.g. by brakes being applied) at least temporarily (during the operation). The multi-joint robot arm extends away from the platform, so that preferably a mounting base, which carries the robot arm, is preferably fixed directly on the platform. As viewed in the longitudinal direction, the fixed end of the multi-joint robot arm, arranged on the platform, lies opposite (i.e. spaced apart from) the free end at which the instrument holder is arranged. The control device is preferably configured to control the multi-joint robot arm in such a way that the free end of the latter is movable in several degrees of freedom (preferably in at least three translational and three rotational degrees of freedom) relative to the platform (or the fixed end). This ensures maximum flexibility of movement, such that the multi-joint robot arm can be adjusted to any desired position and into any desired attitude during an operation.

In another preferred embodiment, the instrument holder is designed as a part of the multi-joint robot arm, in particular as an end effector of the multi-joint robot arm, and the at least one microphone is arranged on the instrument holder. In this embodiment, the microphone is therefore at the outer, distal end of the multi-joint robot arm, which end, viewed along a longitudinal extent of the multi-joint robot arm, is at the furthest distance from its fixed end. This embodiment has the advantage that the instrument holder, as the end effector of the multi-joint robot arm, is located in direct proximity to the medical and/or surgical instrument, which is always placed in direct proximity to the surgeon during an operation. Thus, speech detection is possible in proximity to the surgeon. The instrument holder is preferably formed directly on the multi-joint robot arm. The instrument holder preferably forms the end effector of the robot arm, which end effector adjoins the last joint connection provided before the free end of the robot arm. In robotics, the end effector is the last element of a kinematic chain. The expression “(open) kinematic chains” or “kinematic path” is understood here as meaning that a plurality of arm sections of the multi-joint robot arm are in each case connected to each other by at least one joint. The kinematic path begins at the fixed end of the robot arm, where the latter is mounted, for example, on the platform or similar, and it ends at the free end effector.

In another preferred embodiment, the at least one microphone is arranged on and/or integrated into an arm section and/or a joint section of the multi-joint robot arm. The instrument holder is particularly preferably arranged on the arm section and/or on the joint section directly (without further Intermediate elements) or indirectly (via other intermediate elements). In this embodiment, the at least one microphone, viewed along the kinematic path, is preferably arranged in an arm section and/or a joint section of the multi-joint robot arm located before the end effector. Therefore, in this embodiment, the microphone is not arranged directly on the end effector or on the instrument holder, but before the latter. In this arrangement, the microphone is still located in the end region of the multi-joint robot arm, such that it is still in direct proximity to the surgeon during an operation, thus enabling high-quality speech detection. This arrangement can be advantageous if the instrument holder, as the end effector, is structurally unsuitable for the arrangement and/or integration of the microphone.

In another preferred embodiment, the at least one microphone is covered by a cover, in particular a non-stick cover. The microphone is therefore preferably not arranged exposed on the multi-joint robot arm, and instead it is covered over by the cover during its intended use. For example, the cover can be a membrane, a film or a plastic element, which is permeable to acoustic sound waves (and thus to the voice that is to be detected) but can be sterilized in a conventional manner (e.g. by UV sterilization) without any problems. It is advantageous in this embodiment that the microphone can preferably be designed as a standard component, since it does not itself have to be designed for sterilization. The only thing that needs to be sterilized is the cover that covers the microphone and protects it from the outside. By virtue of this embodiment, the production costs for the speech detection according to the disclosure can be minimized. It is particularly advantageous if the cover is designed as a non-stick cover or at least has a non-stick coating, in order thereby to avoid or at least minimize the accumulation of particles, germs, bacteria and viruses. It can also be advantageous if the cover has antimicrobial properties, which can be provided by a coating, for example.

In a further preferred embodiment, the multi-joint robot arm comprises a plurality of arm sections, which are connected to one another via in each case at least one joint with in each case at least one joint drive, wherein the control device is designed to control each joint drive individually. The multi-joint robot arm therefore preferably comprises at least a first arm section and a second arm section. The first arm section can, for example, be connected via a first joint connection to an aforementioned platform. The first joint connection can preferably allow a mobility of the first arm section relative to the platform with at least one degree of freedom. The first arm section can preferably be connected to the second arm section movably via a second joint connection. The second joint connection can preferably allow a mobility of the second arm section relative to the first arm section with at least one degree of freedom. The second arm section is preferably connected to the instrument holder via a third joint connection. The third joint connection can preferably allow a mobility of the instrument holder relative to the second arm section with at least one degree of freedom. Each of the joint connections preferably has at least one drive, by which the respective joint connection can be moved. Each drive can preferably be controlled independently. It will be appreciated that the robot arm can also have further arm sections and joint connections, and that the respective joint connection can also provide a mobility in several degrees of freedom.

In a further preferred embodiment, the speech detection device comprises a speech recognition device, which is configured to detect at least one information content of a word and/or text spoken by the user, to check this for the presence of a predefined control command, and, if at least one predefined control command is present in the information content, to transmit the at least one control command to the control device. Particularly preferably, the control device is designed to control the multi-joint robot arm and/or the medical and/or surgical instrument on the basis of the transmitted control command. In this way, voice-based control of the multi-joint robot arm and/or of the medical and/or surgical instrument is made possible. This has the advantage that the surgeon and/or a medical assistant can carry out voice-based control. Preferably, a large number of control commands are predefined and, for example, stored in a database in such a way that the speech recognition device can compare the determined information content against the database entries. A control instruction, which can be transmitted to the control device, is preferably stored in the database for each control command. It is possible, for example, that the surgeon pronounces a text during the operation, such as “approach dorsal position”, and the speech recognition device determines from this the information content “dorsal position”, for which a control command exists by which the multi-joint robot arm is caused to approach the predefined, dorsal position on the patient.

In another preferred embodiment, the speech recognition device is configured to authenticate the user on the basis of his or her voice. This embodiment has the advantage that preferably only an authenticated user has permission for voice-assisted control of the at least one medical and/or surgical instrument and/or of the multi-joint robot arm, whereas unauthenticated users are excluded from such control. The authentication is preferably based on a voice profile of the user, which is predefined and thus can be registered by the speech recognition device. For example, if the user speaks with the correct voice profile, the user can be authenticated. In this way, incorrect operation and/or the risk of erroneous or unwanted generation of a control command can be minimized.

In a further preferred embodiment, the multi-joint robot arm comprises at least one sensor which is configured to detect a movement of at least the head or a hand of the user in spatial terms and to transmit this in the form of sensor signals to the control device, wherein the control device is designed to control the multi-joint robot arm on the basis of the sensor signals, in such a way that at least the instrument holder follows the movement of the user's head while maintaining a predetermined minimum distance. The sensor can be a camera, for example. This makes it possible for the multi-joint robot arm to follow the movements of at least the head or at least one of the hands of the surgeon in a sensor-controlled manner, without the surgeon having to initiate an independent control command. This has the advantage that the instrument is always in a predefined proximity (maintaining a predefined minimum distance) to the surgeon or to another user. Thus, the microphone arranged on the multi-joint robot arm is also located in direct proximity to the user with the aid of sensors, so that high-quality speech detection and/or speech recognition is made possible in a particularly comfortable manner. A further advantage is that this embodiment improves the ergonomics and general handling of the handling device according to the disclosure.

The disclosure also includes a medical system which has a handling device according one of the embodiments of the disclosure and a medical and/or surgical instrument which is held on the instrument holder directly (without further intermediate elements) or indirectly (via at least one further intermediate element). It will be appreciated that the features and advantages regarding the exemplary embodiments of the handling device according to the disclosure apply in an equivalent manner to the system according to the disclosure and need not be mentioned again in respect of the latter.

In a preferred embodiment, the medical and/or surgical instrument comprises an exoscope and/or a microscope and/or an endoscope and/or a sterile barrier and/or a cystoscope and/or a laparoscope and/or an arthroscope and/or a sterile adapter and/or an operating-room light. It will be appreciated that it is also possible in principle for a plurality of instruments to be arranged on the instrument holder according to the disclosure. It will also be appreciated that the abovementioned instruments are not to be understood in any way as restrictive and, in principle, other medical and/or surgical instruments and/or aids that are not explicitly mentioned are also included. During an operation, the at least one medical and/or surgical instrument preferably has a predefined distance from a site of the patient and is preferably likewise located at a predefined distance from the surgeon's head. Thus, spoken commands of the surgeon and of other auxiliary personnel can preferably always be detected in proximity to their place of origin. This minimizes the influence of secondary noise.

### BRIEF DESCRIPTION OF THE DRAWINGS

Further advantages and details of the disclosure will become clear from the following description of preferred embodiments of the disclosure and from the drawings, which are merely schematic.

FIG. 1 shows a perspective view of an exemplary embodiment of a handling device according to the disclosure; and

FIG. 2 shows a perspective detailed view of the exemplary embodiment of a handling device according to the disclosure.

The same elements, or elements having the same function, are provided with the same reference numbers in the figures.

### DETAILED DESCRIPTION OF PREFERRED EMBODIMENTS

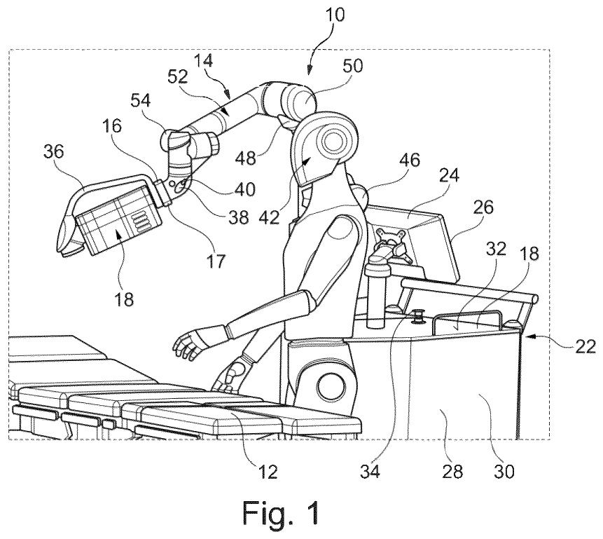

FIG. 1 shows a perspective overview of a handling device 10 according to an exemplary embodiment of the disclosure. During an operation, the handling device 10 is assigned in principle to a patient not shown in detail, who is placed on an operating table 12. The handling device 10 can be used for therapeutic, surgical and/or diagnostic purposes. However, it is also conceivable for it to be used for non-therapeutic, non-surgical and/or non-diagnostic purposes. These can include, among other things, uses for training exercises or simulations.

The handling device 10 comprises a multi-joint robot arm 14, at the distal, free end of which an instrument holder 16 is formed. The instrument holder 16 is designed to carry at least one medical and/or surgical instrument 18 or preferably to fasten the latter reversibly in a releasable or interchangeable manner. The instrument holder 16 forms an end effector 17 of the multi-joint robot arm 14. In the example shown, the medical instrument 18 is an exoscope. In the present case, an exoscope is a high-tech surgical microscope that can be used, for example, to visualize anatomical details of a human or animal brain or another body part in a three-dimensional manner. The exoscope can provide a four-fold resolution (4K), which allows a high depth of field compared to other instruments, which is desirable for observing a particular object field.

For example, alternative embodiments are conceivable in which the handling device 10 can be used for observing the interior of the body, for example with an endoscope or a laparoscope being held by the instrument holder.

The handling device 10 comprises a platform 20, which in the present case is designed as a trolley or carriage 22, of which only an upper section is visible in FIG. 1. This should not be construed as restrictive. The platform 20, for example as the carriage 22, can be designed with a chassis (not shown in detail). The carriage 22 can comprise one or more supports in order to protect the carriage 22 against undesirable movement during use of the handling device 10, i.e. during an operation. Accordingly, such a support can serve to arrange the carriage 22 in a fixed position in the operating room. Alternatively or in addition, the carriage 22 can have wheels and a chassis, as well as a parking brake, in order to enable an adjustment of the carriage 22, and thus also of the platform 20, only if it is ensured that the carriage 22 is not unintentionally movable. It will be appreciated that the carriage 22 can also be anchored/fixed in other ways to enable safe use of the handling device 10.

The multi-joint robot arm 14 extends starting from the platform 20. Also arranged on the platform 20 is a computer terminal 24, which comprises a screen 26 on which a user can, for example, make adjustments to the multi-joint robot arm 14 and/or to the at least one instrument 18. Arranged in the carriage 22, or below a housing 28, is a control device 30 which is configured to control the multi-joint robot arm 14 and the at least one medical and/or surgical instrument 18. The control device 30 can in principle also be integrated in the computer terminal 24 or formed by the latter. Furthermore, on its outer surface 32 facing away from the ground, the platform 22 comprises an emergency stop button 34 which is designed to switch off the multi-joint robot arm 14 and/or the instrument 18, for example in the event of incorrect use, an emergency during the operation and/or another incident. When switched off by the emergency stop button, the multi-joint robot arm 14 preferably moves to a predefined rest position in which it is located outside the operating environment around the operating table 12, so that this region is freely accessible in an emergency.

On the instrument 18 in the present case, a handle 36 is attached, by which the instrument 18 can, if necessary, also be moved manually to a different position above the operating table 12.

The handling device 10 according to the disclosure is characterized in that it comprises a speech detection device 38 with at least one microphone 40. The microphone 40 is arranged and/or integrated in a region of the distal end of the multi-joint robot arm 14 (in a region of the end effector 17 or adjacent to the end effector 17). The speech detection device 38 is designed to detect at least one voice of a user 42 of the handling device 10. The speech detection device 38 can further comprise a speech recognition device 44, which is configured to detect an information content of a text spoken by the user 42, to check this for the presence of at least one predefined control command, and, if the at least one predefined control command is present in the information content, to transmit the control command to the control device 30.

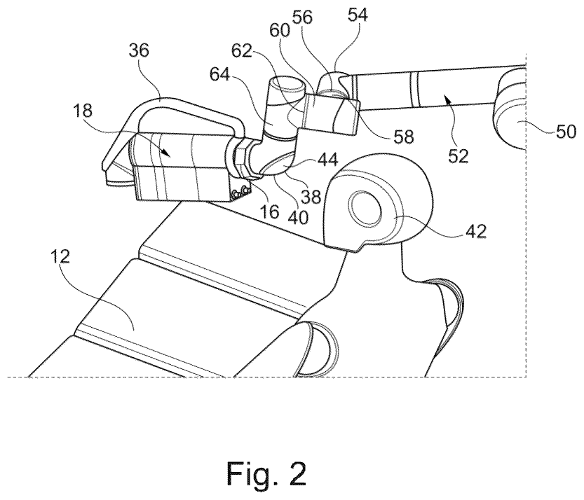

The multi-joint robot arm 14 in the present case comprises a first arm section (not visible) which is arranged on the platform 20 and, starting from the platform 20, extends away from the floor. The first arm section is connected to a second arm section 48 via a controllable joint 46, wherein the first controllable joint 46 allows a movement of the second arm section 48 relative to the first arm section in a rotational degree of freedom. The second arm section 48 is connected to a third arm section 52 via a second controllable joint 50. The second controllable joint 50 allows a movement of the third arm section 52 relative to the second arm section 48 in a rotational degree of freedom. The third arm section 52 is connected to a fourth arm section 56 via a third controllable joint 54. The third controllable joint 54 allows a movement of the fourth arm section 56 relative to the third arm section 52 in a rotational degree of freedom. The fourth arm section 56 is connected to a fifth arm section 60 via a fourth controllable joint 58. The fourth controllable joint 58 allows a movement of the fifth arm section 60 relative to the fourth arm section 56 in a rotational degree of freedom. The fifth arm section 60 is connected to a sixth arm section 64 via a fifth controllable joint 62. The fifth controllable joint 62 allows a movement of the sixth arm section 64 relative to the fifth arm section 60 in a rotational degree of freedom. The instrument holder 16 is arranged directly adjacent to the sixth arm section 64 and thus forms the free, distal end effector 17. In the present case, the exoscope is arranged on the instrument holder 16. Each of the joints 46, 50, 54, 58, 62 can be individually controlled by the control device 30 and for this purpose each has a drive, for example in the form of an electric motor. In the present case, the sixth arm section 64 is designed as a curved tube. In the present case, the at least one microphone 40 is arranged in or integrated into the distal sixth arm section 64 at the end of the multi-joint robot arm 14. The respective joint drive is preferably integrated in the form of an electric actuator in each of the joints 46, 50, 54, 58, 62. For a detailed view of the multi-joint robot arm 14, reference may be made to FIG. 2.

The microphone 40 is covered there by a (preferably non-stick and/or antimicrobial) cover 66, so that simple sterilization of the microphone 40 can be made possible.

1. A handling device for handling at least one medical and/or surgical instrument, comprising:
a multi-joint robot arm, which at its distal end carries an instrument holder designed for holding a medical and/or surgical instrument, and
a control device, which is designed at least to control the multi-joint robot arm, characterized in that
the handling device comprises a speech detection device with at least one microphone, which is arranged in a region of the distal end of the multi-joint robot arm and/or integrated therein and is designed to detect at least a voice of a user of the handling device.

2. The handling device as claimed in claim 1, wherein the handling device comprises a platform from which the multi-joint robot arm extends, and the control device is designed to move at least the distal end relative to the platform.

3. The handling device as claimed in claim 1 wherein, the instrument holder is designed as part of the multi-joint robot arm, in particular as an end effector of the multi-joint robot arm, and the at least one microphone is arranged on the instrument holder.

4. The handling device as claimed in claim 1, wherein, the at least one microphone is arranged on or integrated in an arm section and/or a joint of the multi-joint robot arm on which the instrument holder is directly or indirectly arranged.

5. The handling device as claimed in claim 1, wherein, the at least one microphone is covered by a cover, in particular a non-stick cover.

6. The handling device as claimed in claim 1,
the multi-joint robot arm has a plurality of arm sections, which are connected to one another in each case via at least one joint with at least one joint drive, wherein the control device is designed to control each joint drive individually.

7. The handling device as claimed in claim 1, wherein, the speech detection device comprises a speech recognition device, which is designed to detect an information content of a text spoken by the user, to check this for the presence of at least one predefined control command, and, if the at least one predefined control command is present in the information content, to transmit the control command to the control device .

8. The handling device as claimed in claim 1, wherein, the speech recognition device is configured to authenticate the user on the basis of his or her voice.

9. The handling device as claimed in claim 1, wherein, characterized in that-the multi-joint robot arm comprises at least one sensor which is configured to detect a movement of at least the head of the user in spatial terms and to transmit this in the form of sensor signals to the control device, wherein the control device is designed, on the basis of the sensor signals, to control the multi-joint robot arm in such a way that at least the instrument holder follows the movement of the head of the user while maintaining a predetermined minimum distance.

10. A medical system comprising a handling device as claimed in claim 1 and a medical and/or surgical instrument that is directly or indirectly held on the instrument holder.

11. The medical system as claimed in claim 10, wherein that the medical and/or surgical instrument comprises an exoscope and/or a microscope and/or an endoscope and/or a sterile barrier and/or a cystoscope and/or a laparoscope and/or an arthroscope and/or a sterile adapter and/or an operating-room light.
